(19) United States
(12) Patent Application Publication
(10) Pub. No.: US 2016/0015372 A1
(43) Pub. Date: Jan. 21, 2016

(54) EXPANDING BIOPSY CATHETER

(71) Applicant: Boston Scientific Scimed, Inc., Maple Grove, MN (US)

(72) Inventors: Steven E. Walak, Natick, MA (US); Michael Powers, Pepperell, MA (US)

(21) Appl. No.: 14/789,547

(22) Filed: Jul. 1, 2015

Related U.S. Application Data
(60) Provisional application No. 62/024,760, filed on Jul. 15, 2014.

(51) Int. Cl. A61B 10/02; A61B 10/04

(52) U.S. Cl. CPC A61B 10/0266; A61B 10/04

(57) ABSTRACT

A device for collecting a tissue sample includes an outer sheath extending longitudinally from a proximal end to a distal end and including a lumen extending therethrough. The device also includes a tissue collecting member movably housed within the lumen and biased toward an expanded configuration in which the tissue collecting member is curved about a longitudinal axis thereof and longitudinal edges thereof are separated from one another. The tissue collecting member is movable between a constrained position, in which the tissue collecting is received within the lumen and constrained by a surface thereof such that longitudinal edges of the tissue collecting member are drawn toward one another to define a channel therein, and a tissue collecting position, in which the tissue collecting member moves distally past the distal end of the outer sheath to revert to the biased expanded configuration to cut a target tissue into which it is inserted.

### PRIORITY INFORMATION

The present invention claims priority to U.S. Provisional Patent Application Ser. No. 62/024,760 filed Jul. 15, 2014; the disclosure of which is incorporated herewith by reference.

### BACKGROUND

Needle biopsy procedures are common for the diagnosis and the staging of disease. For example, a fine needle aspiration needle may be advanced through a working channel of an endoscope to a target tissue site. Although fine needle aspiration is a highly sensitive and specific procedure, it may be difficult to acquire a suitable sample under certain clinical situations. The more cells or tissue that can be acquired, the greater the potential for a definitive diagnosis. Larger gauge needles, however, may be difficult to pass along tortuous paths through anatomy to target sites and may acquire samples including more blood, making it more difficult to obtain a diagnosis.

### SUMMARY

The present disclosure is directed to a device for collecting a tissue sample, comprising an outer sheath extending longitudinally from a proximal end to a distal end and including a lumen extending therethrough and a tissue collecting member movably housed within the lumen of the outer sheath and being biased toward an expanded configuration in which the tissue collecting member is curved about a longitudinal axis thereof and longitudinal edges thereof are separated from one another, the tissue collecting member movable between a constrained position, in which the tissue collecting member is received within the lumen and constrained by a surface thereof such that longitudinal edges of the tissue collecting member are drawn toward one another to define a channel therein, and a tissue collecting position, in which the tissue collecting member moves distally past the distal end of the outer sheath to revert to the biased expanded configuration such that the longitudinal edges and a distal edge of the tissue collecting member are adapted to cut a target tissue into which it is inserted.

In an embodiment, the device may further comprise a control element extending longitudinally from a proximal end which, when the device is inserted to a target area within a patient body, is adapted to remain external to the patient body to remain accessible to a user, to a distal end connected to the tissue collecting member.

In an embodiment, longitudinal and distal edges of the tissue collecting member may be sharpened to facilitate cutting of the target tissue.

In an embodiment, the longitudinal and distal edges of the tissue collecting member may be serrated to facilitate cutting of the target tissue.

In an embodiment, the longitudinal edges may one of contact and overlap one another to define the channel.

In an embodiment, the tissue collecting member may be curved relative to the longitudinal axis thereof such that the tissue collecting member forms a cupping shape.

In an embodiment, the device may further comprise a stylet extending longitudinally from a proximal end to a distal tip, the stylet extending along the longitudinal axis so that, when the tissue collecting member is in the constrained position, the distal tip extends distally past the distal edge of the tissue collecting member to pierce the target tissue into which it is inserted.

In an embodiment, the stylet may include barbs along a portion of a length thereof for gripping tissue cut by the tissue collecting member as the device is withdrawn from the target tissue in the constrained position.

In an embodiment, the stylet may include a roughened surface for gripping tissue cut by the tissue collected member as the device is withdrawn from the target tissue in the constrained position.

In an embodiment, an interior surface of the tissue collecting member may include inwardly projecting teeth for gripping target tissue collected within the channel defined thereby in the constrained configuration.

In an embodiment, the tissue collecting member may be formed from a nitinol sheet annealed at temperature to create a superelastic material biased toward the expanded configuration.

The present disclosure is also directed to a device for collecting a target tissue, comprising an outer sheath extending longitudinally from a proximal end to a distal end and including a lumen extending therethrough, a tissue collecting member movably housed within the lumen of the outer sheath and being biased toward an expanded configuration in which the tissue collecting member is curved about a longitudinal axis thereof and longitudinal edges thereof are separated from one another, the tissue collecting member movable between a constrained position, in which the tissue collecting member is received within the lumen and constrained by a surface thereof such that longitudinal edges of the tissue collecting member are drawn toward one another to define a channel therein, and a tissue collecting position, in which the tissue collecting member moves distally past the distal end of the outer sheath to revert to the biased expanded configuration such that the longitudinal edges and a distal edge of the tissue collecting member are adapted to cut a target tissue into which it is inserted, and a control member extending longitudinally from a proximal end to a distal end connected to the tissue collecting member for moving the tissue collecting member longitudinally relative to the outer sheath.

In an embodiment, the outer sheath may be formed of one of a metal tubing and a polymer composite braided structure.

In an embodiment the device may further comprise a stylet extending longitudinally from a proximal end to a distal tip, the stylet extending along the longitudinal axis so that, when the tissue collecting member is in the constrained position, the distal tip extends distally past the distal edge of the tissue collecting member to pierce the target tissue into which it is inserted.

In an embodiment, the tissue collecting member may be formed from a nitinol sheet annealed at temperature to create a superelastic material biased toward the expanded configuration.

In another aspect, the present disclosure is also directed to a method for collecting a tissue sample, comprising inserting an outer sheath through a working channel of an endoscope until a distal end of the outer sheath is proximally adjacent a target tissue, inserting a tissue collecting member into the target tissue by moving the tissue collecting member distally relative to the outer sheath in which it is received so that the tissue collecting member extends distally past a distal end of the outer sheath and is permitted to revert to an expanded biased configuration in which the tissue collecting member is curved about a longitudinal axis thereof such that longitudinal edges thereof are separated from one another, wherein the tissue collecting member cuts the target tissue as it is inserted into the target tissue, and moving the tissue collecting member distally over the tissue collecting member so an interior surface of the outer sheath constrains the tissue collecting member, causing the longitudinal edges of the tissue collecting member to be drawn toward one another to define a channel thereby in which the target tissue is trapped.

### BRIEF DESCRIPTION OF THE DRAWINGS

FIG. 1 shows a perspective view of a device according to an exemplary embodiment of the present disclosure, in a tissue collecting position;

FIG. 2 shows a perspective view of the device of FIG. 1, in a constrained position;

FIG. 3 shows a perspective view of a device according to another exemplary embodiment of the present disclosure, in a tissue collecting position;

FIG. 4 shows a perspective view of the device of FIG. 3, in a constrained position;

FIG. 5 shows a perspective view of a device according to yet another exemplary embodiment of the present disclosure;

FIG. 6 shows a top plan view of a tissue collecting member of the device of FIG. 5;

FIG. 7 shows a perspective view of a device according to another exemplary embodiment of the present disclosure, in a tissue collecting position;

FIG. 8 shows a perspective view of the device of FIG. 7, in a constrained position;

FIG. 9 shows a perspective view of a device according to another exemplary embodiment of the present disclosure;

FIG. 10 shows a perspective view of a device according to yet another exemplary embodiment of the present disclosure;

FIG. 11 shows a side view of a device according to another exemplary embodiment, in an unexpanded configuration;

FIG. 12 shows a side view of the device of FIG. 11, in an expanded configuration;

FIG. 13 shows a side view of a device according to another exemplary embodiment of the present invention, in an expanded configuration; and

FIG. 14 shows a side view of the device of FIG. 13, in an unexpanded configuration.

### DETAILED DESCRIPTION

The present disclosure may be further understood with reference to the following description and the appended drawings, wherein like elements are referred to with the same reference numerals. The present disclosure relates to endoscopic devices and, in particular, devices for obtaining tissue samples. Exemplary embodiments of the present disclosure describe a device comprising a tissue collecting member that expands as it is moved distally out of an outer sheath to collect a larger tissue sample than would be possible with traditional needle biopsy devices. It should be noted that the terms “proximal” and “distal” as used herein, are intended to refer to a direction toward (proximal) and away from (distal) a user of the device.

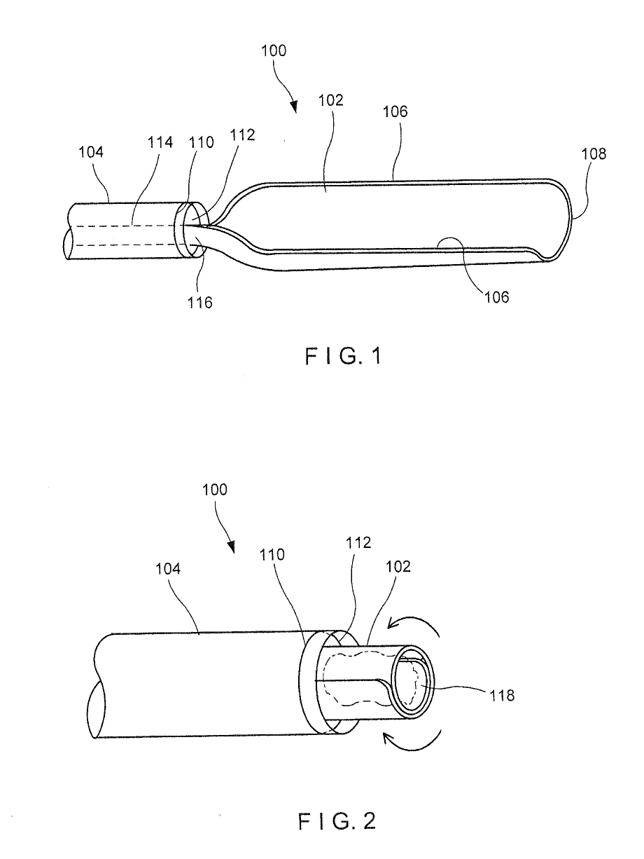

As shown in FIGS. 1-2, a device 100 according to an exemplary embodiment of the present disclosure comprises an expandable tissue collecting member 102 movably housed within an outer sheath 104. The tissue collecting member 102 is biased toward an expanded state so that the device 100 is movable between a constrained position, in which the tissue collecting member 102 is received within the outer sheath 104 and constrained to remain in an unexpanded state by contact with an inner surface of the outer sheath 104, and a tissue collecting position, in which the tissue collecting member 102 is moved distally with respect to the outer sheath 104 such that the tissue collecting member 102 is no longer constrained by the outer sheath 104 and is permitted to revert under natural bias to the expanded state. The tissue collecting member 102 may be moved relative to the outer sheath 104 via a control element 114 (e.g., a flexible wire with column strength sufficient to advance the member 102 through the outer sheath 104) extending from a proximal end to a distal end 116 connected to the tissue collecting member 102. The tissue collecting member 102 may be configured as a metal sheet curved about a longitudinal axis thereof such that longitudinal edges 106 thereof are separated from one another in the expanded position and drawn toward one another in the constrained position to reduce a radius of curvature of the tissue collecting member 102. All or portions of the longitudinal edges 106 and a distal edge 108 are sharpened to be configured as cutting edges so that the tissue collecting member 102 cuts tissue as the device 100 is moved to the tissue collecting position to separate a tissue sample from surrounding tissue. Once the target tissue has been cut, the outer sheath 104 may be moved distally over the tissue collecting member 102, compressing the tissue collecting member 102 back to the constrained position, with the target tissue trapped therein. The proximal ends of the outer sheath 104 and the control element 114 may be connected to a handle assembly which remains accessible to a user, when the device 100 is inserted to a target tissue within a patient body via, for example, a working channel of an endoscope. The handle assembly may include actuators for moving the tissue collecting member 102 and the outer sheath 104 relative to one another.

The outer sheath 104 extends longitudinally from a proximal end (not shown) to a distal end 110 and includes a lumen 112 extending therethrough. A lateral cross-sectional area of the sheath 104 is sized and shaped for insertion through an insertion device such as through a working channel of an endoscope. The outer sheath 104, or at least a distal portion thereof in which the tissue collecting member 102 is constrained, may be formed to exhibit an increased stiffness and hoop strength to facilitate constraint of the expandable tissue collecting member 102. In one exemplary embodiment, the outer sheath 104 may be formed of stainless steel or nitinol. In another embodiment, the outer sheath 104 may be formed of a high stiffness polymer such as PEEK or a composite structure. The composite outer sheath 104 may be comprised of for example, high strength filaments oriented at an acute angle relative to the longitudinal axis of the outer sheath 104 embedded in a polymer matrix to increase circumferential strength of the outer sheath 104. The high strength filaments may be stainless steel wires or ribbon braided or coiled to form a tubular structure. The reinforcing filaments may be metal, carbon, glass, boron, Kevlar, polyethylene terephthalate, Ultra-High Molecular Weight Polyethylene (UHMwPE) or another suitable high strength material.

The tissue collecting member 102 may be fabricated from, for example, a flat nitinol sheet. The sheet may be cut to a desired shape and size, formed into the expanded coil configuration and heat treated (e.g., by annealing) at a temperature selected to create a memorized shape of a superelastic material (e.g., Nitinol) and to permanently set this memorized shape in the expanded coil configuration. The sheet may then be connected to the distal end 116 of the control element 114 by, for example, welding or mechanical fixation. Alternatively, the control element 114 may be integrally formed therewith. A thickness, shape and size of the tissue collecting member 102 may be selected to provide sufficient longitudinal stiffness for distal penetration of tissue with minimum outward expansion force. It will be understood by those of skill in the art that minimizing outward expansion force reduces hoop strength requirements for the outer sheath 104.

In the tissue collecting position, the tissue collecting member 102 maintains the enlarged coil configuration, in which the sheet is curved about a longitudinal axis of the device 100. When the tissue collecting member 102 is received within the lumen 112 of the outer sheath 104 and held thereby in the constrained position with the radius of curvature of the tissue collecting member 102 reduced by drawing longitudinal edges 106 of the tissue collecting member 102 toward one another about the longitudinal axis. In the constrained position, the longitudinal edges 106 may contact one another or overlap one another such that tissue may be entirely trapped within a channel 118 defined by the tissue collecting element 102 in the constrained position, permitting the target tissue to be withdrawn from the patient body via a smaller lumen. Compression of the target tissue within the channel 118 may also aid in expelling blood and other fluids from the target tissue for a more efficient histological analysis thereof. The longitudinal edges 106 and a distal edge 108 of the tissue collecting member 102 (e.g., a distal-most edge of the coiled sheet extending transversely relative to a longitudinal axis of the device 100) may be configured as cutting edges which may be sharpened to facilitate a cutting of the target tissue into which it is inserted.

According to an exemplary surgical technique using the device 100, the device 100 is inserted to a target area within a living body, for example, through a body lumen accessed via a naturally occurring body orifice. The device 100 may be inserted through the working channel of an endoscope or through any other suitable rigid or flexible insertion instrument as would be understood by those skilled in the art. The device 100 may be inserted through the working channel, in the constrained position, until the device 100 is adjacent to target tissue to be sampled. The tissue collecting member 102 is then moved distally with respect to the outer sheath 104 to the tissue collecting position such that the tissue collecting member 102 is inserted into the target tissue. As the tissue collecting member 102 is moved distally out of the outer sheath 104, the tissue collecting member 102 is freed to revert to the biased or memorized expanded state, cutting the target tissue in the process. The tissue collecting member 102 may also be rotated about a longitudinal axis thereof to facilitate the collection of tissue. Once the tissue collecting member 102 has been fully inserted into the target tissue with a sample within the collecting member 102, the device 100 may be returned to the constrained position by moving the outer sheath 104 distally over the tissue collecting member 102. As the outer sheath 104 moves distally over the tissue collecting member 102, the surface of the lumen 112 compresses the tissue collecting member 102 radially inward, drawing longitudinal edges 106 of the tissue collecting member 102 toward one another. The longitudinal edges 106 continue to cut the target tissue as they are drawn toward one another to separate the target tissue from the surrounding tissue and to trap this target tissue in the channel 118 formed thereby. The device 100 may then be removed from the patient body, in the constrained position, with the target tissue trapped therein.

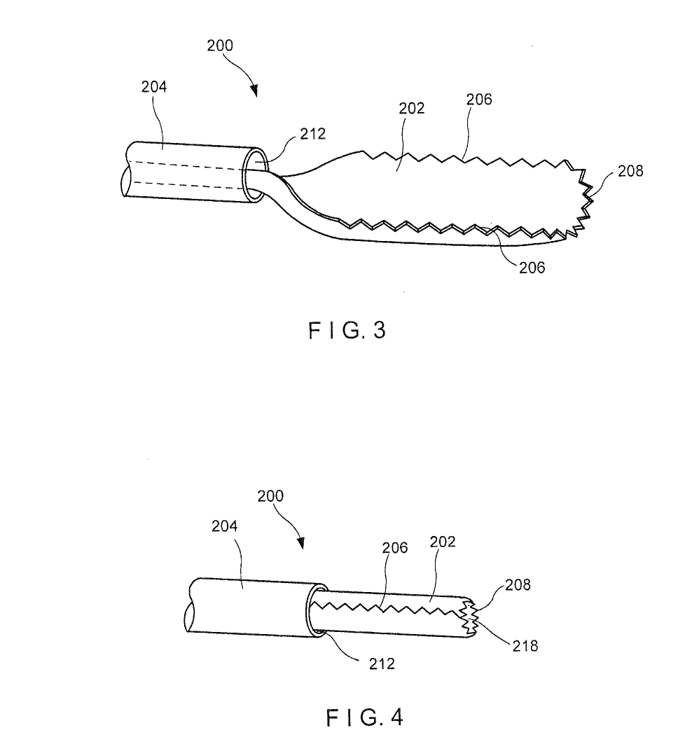

As shown in FIGS. 3-4, a device 200 according to another exemplary embodiment of the present disclosure may be substantially similar to the device 100 described above, comprising a tissue collecting member 202 movably housed within an outer sheath 204 to be moved between a constrained position, in which tissue collecting member 202 is held in an unexpanded state via a surface of a lumen 212 of the outer sheath 204, and a tissue collecting position, in which the tissue collecting member 202 is permitted to revert to its biased expanded state. The tissue collecting member 202 may be substantially similar to the tissue collecting member 102 of the device 100, being formed of a metal sheet curved about a longitudinal axis thereof, in the expanded state. In the constrained position, longitudinal edges 206 of the tissue collecting member 202 are drawn toward one another. Similarly to the tissue collecting member 102, longitudinal and distal edges 206, 208 of the tissue collecting member 202 are configured as cutting edges. The longitudinal and distal edges 206, 208, however, are serrated to facilitate cutting of the target tissue.

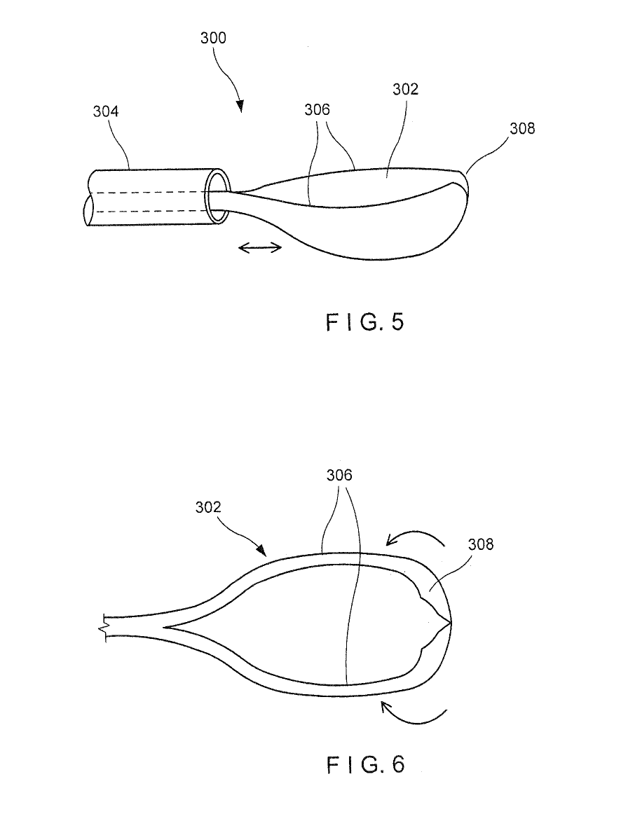

As shown in FIGS. 5-6, a device 300 may be substantially similar to the devices 100, 200, as described above, comprising a tissue collecting member 302 movably housed within an outer sheath 304 between a constrained position and an expanded tissue collecting position. The tissue collecting member 302 may be substantially similar to the tissue collecting member 102, being formed of a coiled or curved metal sheet defined by longitudinal and distal cutting edges 306, 308. The tissue collecting member 302, however, is also curved along the longitudinal axis thereof such that the distal edge 308 is curved toward the longitudinal axis. The tissue collecting member 302 may be substantially spoon-shaped so that a tissue sample may be received within a concave recess defined thereby. The curved distal edge 308 facilitates a severing of an end of the target tissue from a surrounding tissue for easy removal. Thus, in the constrained position, the distal edge 308 substantially closes a distal end of the tissue collecting member 302 as longitudinal edges 306 are moved toward one another. Although the tissue collecting member 302 is shown as having substantially smooth longitudinal and distal edges 306, 308, it will be understood by those of skill in the art that the longitudinal and distal edges 306, 308 may also be serrated, as described above in regard to the tissue collecting member 202 of the device 200.

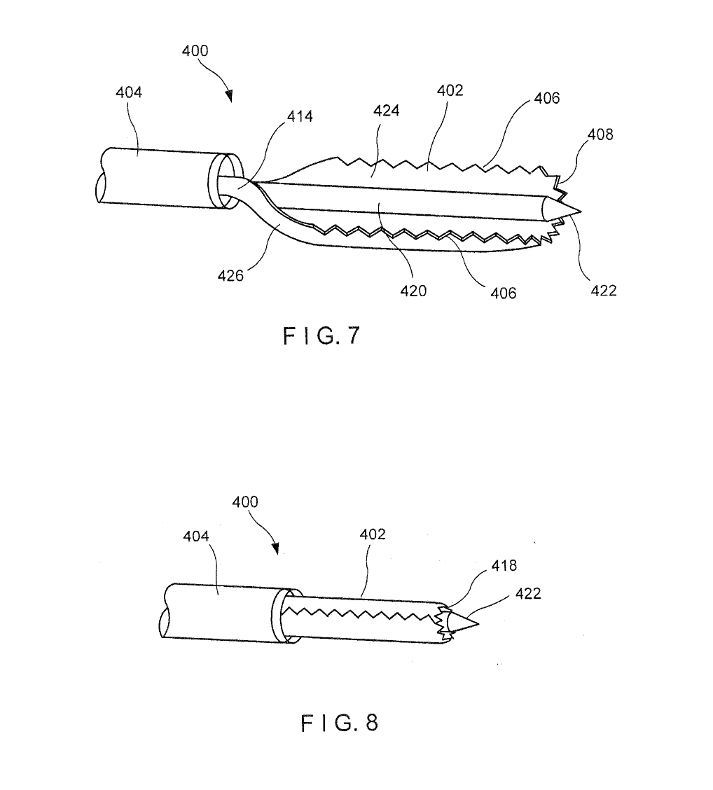

As shown in FIGS. 7-8, a device 400 according to another exemplary embodiment of the present disclosure may be substantially similar to the devices 100-300 described above, comprising a tissue collecting member 402 movably housed within an outer sheath 404 between a constrained position and an expanded tissue collecting position. The device 400, however, further comprises a stylet 420 extending longitudinally from a proximal end to a sharpened distal tip 422. The stylet 420 may be received within a lumen of a control element 414, which is connected to a proximal end 426 of the tissue collecting member 402 such that the stylet 420 extends along the longitudinal axis of the tissue collecting member 402. The stylet 420 may be configured to move independently of the tissue collecting member 402 and the outer sheath 404 or it may be attached to the tissue collecting member 402 or the control member 414. In the expanded tissue collecting position, the stylet 420 extends along an interior surface 424 of the tissue collecting member 402. In the constrained position, the tissue collecting member 402 is wrapped around the stylet 420 to enclose the stylet 420 in a channel 418 defined thereby. The distal tip 422 may extend distally of a distal edge 408 of the tissue collecting member 402 to facilitate distal advancement of the device 400 to a target area within the patient body, in the constrained position. It will be understood by those of skill in the art that the stylet 420 also prevents tissue from entering a channel defined by the tissue collecting member 402, in the constrained position, as the device 400 is inserted into the body. Although longitudinal and distal edges 406, 408 of the tissue collecting member 402 are shown as including serrations, it will be understood by those of skill in the art that the longitudinal and distal edges 406, 408 of the tissue collecting member may also be substantially smooth, as described and shown in regard to the tissue collecting member 102 of the device 100.

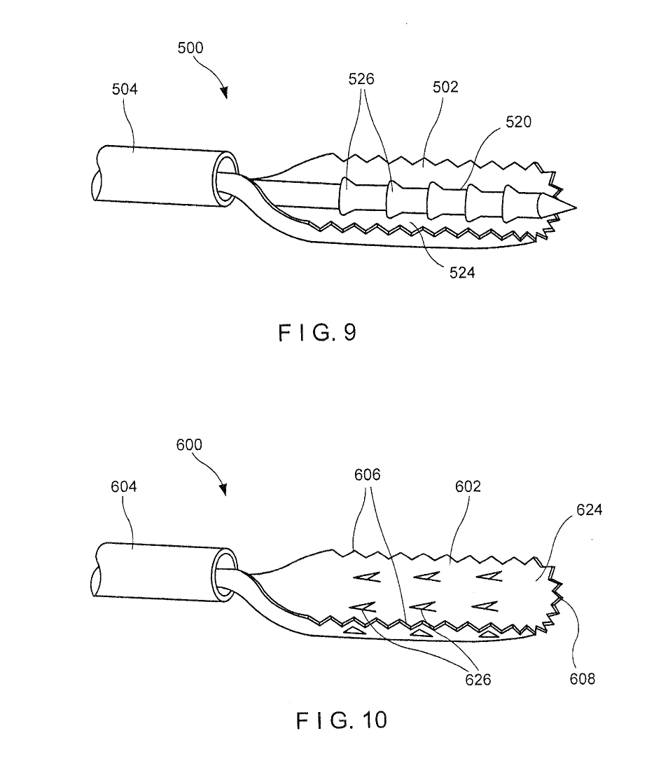

As shown in FIG. 9, a device 500 may be substantially similar to the device 400 described above, comprising a tissue collecting member 502 movably housed in an outer sheath 504 with a stylet 520 extending longitudinally along an interior surface 524 thereof. The tissue collecting member 502 is movable between an expanded tissue collecting position and a constrained position in which longitudinal edges 506 of the tissue collecting member 502 are drawn toward one another to trap target tissue in a channel defined by the tissue collecting member 502, about the stylet 520. The stylet 520, however, may include barbs 526 or a roughened surface along a portion of a length thereof for holding tissue therein during removal of the device 500 from a patient body.

As shown in FIG. 10, a device 600 may be substantially similar to the devices 100, 200, as described above, comprising a tissue collecting member 602 movably housed within an outer sheath 604 to be moved between a constrained position and an expanded tissue collecting position. The tissue collecting member 602, however, may further include teeth 626 projecting inward from an inner surface 624 thereof so that when the device 600 is moved to the constrained position in which longitudinal edges 606 of the tissue collecting member 602 are drawn toward one another to trap a target tissue therein, the teeth 626 grip the target tissue as the device 600 is withdrawn from the patient body. It will be understood by those of skill in the art, however, that the inner surface 624 of the tissue collecting member 602 may include any of a number of features for gripping the target tissue as it is removed from the body. Although longitudinal and distal edges 606, 608 of the tissue collecting member 602 are shown as being serrated, it will be understood by those of skill in the art that the longitudinal and distal edges 606, 608 may also be substantially smooth, as described in regard to tissue collecting member 102 of the device 100.

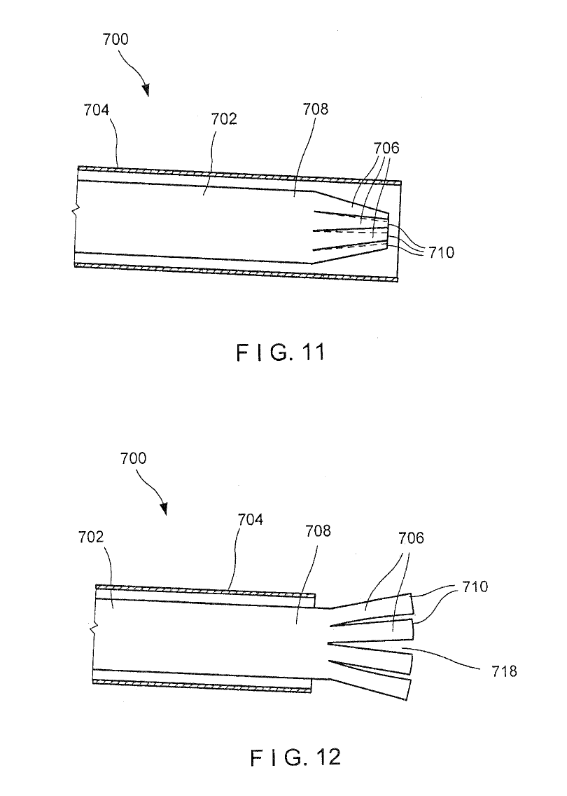

As shown in FIGS. 11-12, a device 700 according to another exemplary embodiment of the present disclosure comprises a tissue collecting member 702 movably housed within an outer sheath 704 to be moved between a position in which the tissue collecting member 702 is held in an unexpanded state via a surface of a lumen of the outer sheath 704, and a tissue collecting position, in which the tissue collecting member 702 is permitted to revert to its biased expanded state. The tissue collecting member 702 may include a plurality of blades 706 at a distal end 708 thereof. The tissue collecting member 702 may extend from a proximal end to the distal end 708 and include a channel 718 extending therethrough. The tissue collecting member 702 is movable between the unexpanded state, in which the blades 706 overlap to form a tapered tip, and the expanded state, in which the distal ends 710 of the blades 706 extend radially away from one another to increase a size of the channel 718 therewithin for tissue acquisition. In the unexpanded state, the outer sheath 704 extends distally over the blades 706 to constrain the blades 706 in the overlapping tapered configuration. Movement of the outer sheath 704 over the blades 706 naturally constrains the blades 706 to maintain them in the unexpanded state. For example, a diameter of the blades 706 is selected to be larger than a diameter of the lumen of the outer sheath 704 so that as the outer sheath 704 is moved over the blades 706, the blades 706 naturally overlap to form the tapered tip. In the expanded stated, the outer sheath 704 is moved proximally of the blades 706 so that the blades 706 are permitted to revert to the biased radially outward configuration.

Edges 710 of the blades 706 are configured as cutting edges and may be sharpened to facilitate cutting tissue into which they are inserted. Target tissue may be drawn into the channel 718 via, for example, a suction force through the channel 718 or a needle extending distally therethrough. Once the target tissue has been received within the channel 718, the tissue collecting member 702 may be moved to the unexpanded state, drawing the distal ends 710 of the blades toward one another so that the blades 706 overlap, thereby cutting the target tissue from surrounding tissue.

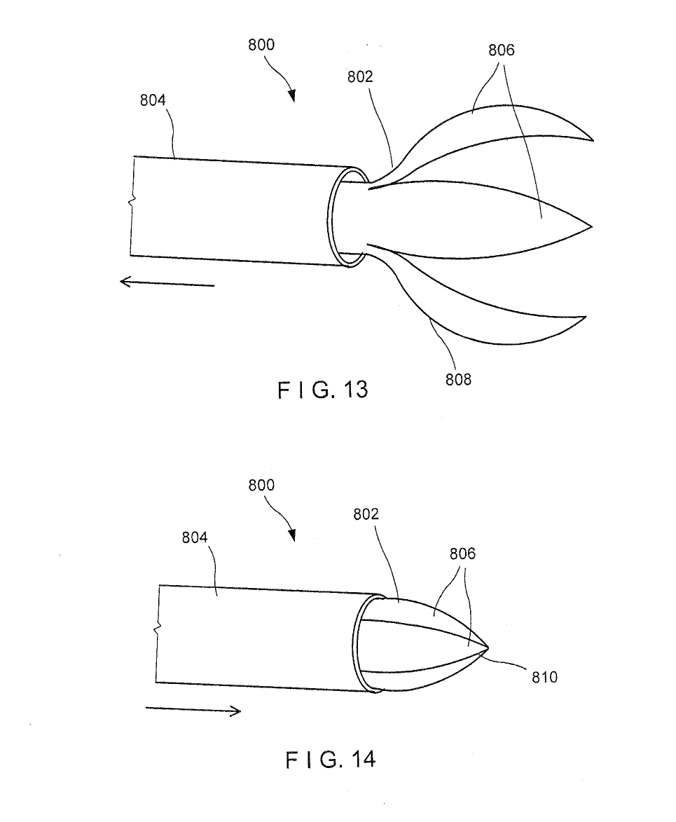

As shown in FIGS. 13-14, a device 800 according to another exemplary embodiment of the present disclosure is substantially similar to the device 700 described above, comprising a tissue collecting member 802 slidably received within an outer sheath 804 to be moved between an expanded state and an unexpanded state. The tissue collecting member 802, however, includes a plurality of curved blades 806 at a distal end 808 thereof so that when the outer sheath 804 is moved distally thereover, an interior surface of the outer sheath 804 contacts exterior surfaces of the curved blades 806 to move the tissue collecting member 802 from an expanded state, in which the curved blades 806 extend radially outward relative to a longitudinal axis of the device 800, to an unexpanded state, in which the curved blades 806 are moved radially inward to cut and collect tissue within a portion of a channel 808 defined thereby. The curved blades 806 may be shaped so that, when the blades 806 are moved to the unexpanded state, the blades 806 contact one another to form a tapered tip 810.

It will be apparent to those skilled in the art that variations can be made in the structure and methodology of the present disclosure, without departing from the scope of the disclosure.

Thus, it is intended that the present disclosure cover the modifications and variations of this disclosure provided that they come within the scope of the appended claims and their equivalents.

1-15. (canceled)

16. A device for collecting a tissue sample, comprising:
an outer sheath extending longitudinally from a proximal end to a distal end and including a lumen extending therethrough; and
a tissue collecting member movably housed within the lumen of the outer sheath and being biased toward an expanded configuration in which the tissue collecting member is curved about a longitudinal axis thereof and longitudinal edges thereof are separated from one another, the tissue collecting member movable between a constrained position, in which the tissue collecting member is received within the lumen and constrained by a surface thereof such that longitudinal edges of the tissue collecting member are drawn toward one another to define a channel therein, and a tissue collecting position, in which the tissue collecting member moves distally past the distal end of the outer sheath to revert to the biased expanded configuration such that the longitudinal edges and a distal edge of the tissue collecting member cut a target tissue into which it is inserted.

17. The device of claim 16, further comprising a control element extending longitudinally from a proximal end which, when the device is inserted to a target area within a patient body remains external to the patient body to remain accessible to a user, to a distal end connected to the tissue collecting member.

18. The device of claim 16, wherein the longitudinal and distal edges of the tissue collecting member are sharpened to facilitate cutting of the target tissue.

19. The device of claim 16, wherein the longitudinal and distal edges of the tissue collecting member are serrated to facilitate cutting of the target tissue.

20. The device of claim 16, wherein the longitudinal edges one of contact and overlap one another to define the channel.

21. The device of claim 16, wherein the tissue collecting member is curved relative to the longitudinal axis thereof such that the tissue collecting member forms a cupping shape.

22. The device of claim 16, further comprising a stylet extending longitudinally from a proximal end to a distal tip, the stylet extending along the longitudinal axis so that, when the tissue collecting member is in the constrained position, the distal tip extends distally past the distal edge of the tissue collecting member to pierce the target tissue into which it is inserted.

23. The device of claim 22, wherein the stylet includes barbs along a portion of a length thereof for gripping tissue cut by the tissue collecting member as the device is withdrawn from the target tissue in the constrained position.

24. The device of claim 22, wherein the stylet includes a roughened surface for gripping tissue cut by the tissue collecting member as the device is withdrawn from the target tissue in the constrained position.

25. The device of claim 16, wherein an interior surface of the tissue collecting member includes inwardly projecting teeth for gripping target tissue collected within the channel defined thereby in the constrained configuration.

26. The device of claim 16, wherein the tissue collecting member is formed from a nitinol sheet annealed at temperature to create a superelastic material biased toward the expanded configuration.

27. A device for collecting a target tissue, comprising:
an outer sheath extending longitudinally from a proximal end to a distal end and including a lumen extending therethrough;
a tissue collecting member movably housed within the lumen of the outer sheath and being biased toward an expanded configuration in which the tissue collecting member is curved about a longitudinal axis thereof and longitudinal edges thereof are separated from one another, the tissue collecting member movable between a constrained position, in which the tissue collecting member is received within the lumen and constrained by a surface thereof such that longitudinal edges of the tissue collecting member are drawn toward one another to define a channel therein, and a tissue collecting position, in which the tissue collecting member moves distally past the distal end of the outer sheath to revert to the biased expanded configuration such that the longitudinal edges and a distal edge of the tissue collecting member cut a target tissue into which it is inserted; and
a control member extending longitudinally from a proximal end to a distal end connected to the tissue collecting member for moving the tissue collecting member longitudinally relative to the outer sheath.

28. The device of claim 27, wherein the outer sheath is formed of one of a metal tubing and a polymer composite braided structure.

29. The device of claim 27, further comprising a stylet extending longitudinally from a proximal end to a distal tip, the stylet extending along the longitudinal axis so that, when the tissue collecting member is in the constrained position, the distal tip extends distally past the distal edge of the tissue collecting member to pierce the target tissue into which it is inserted.

30. The device of claim 27, wherein the tissue collecting member is formed from a nitinol sheet annealed at temperature to create a superelastic material biased toward the expanded configuration.

31. A method for collecting a tissue sample, comprising:
inserting an outer sheath through a working channel of an endoscope until a distal end of the outer sheath is proximally adjacent a target tissue;
inserting a tissue collecting member into the target tissue by moving the tissue collecting member distally relative to the outer sheath in which it is received so that the tissue collecting member extends distally past a distal end of the outer sheath and is permitted to revert to an expanded biased configuration in which the tissue collecting member is curved about a longitudinal axis thereof such that longitudinal edges thereof are separated from one another, wherein the tissue collecting member cuts the target tissue as it is inserted into the target tissue; and
moving the tissue collecting member distally over the tissue collecting member so an interior surface of the outer sheath constrains the tissue collecting member, causing the longitudinal edges of the tissue collecting member to be drawn toward one another to define a channel thereby in which the target tissue is trapped.

32. The method of claim 31, wherein the longitudinal and distal edges of the tissue collecting member are sharpened to cut the target tissue.

33. The method of claim 31, wherein the longitudinal and distal edges of the tissue collecting member are serrated to cut the target tissue.

34. The method of claim 31, wherein a distal tip of a stylet extends distally past the distal edge of the tissue collecting member to pierce the target tissue as the tissue collecting member is inserted into the target tissue.

35. The method of claim 31, wherein the target tissue trapped in the channel defined by the tissue collecting member is gripped by teeth projecting inward from an interior surface of the tissue collecting member.
